(19) United States
(12) Patent Application Publication
(10) Pub. No.: US 2009/0142292 A1
(43) Pub. Date: Jun. 4, 2009

(54) Method For The Mitigation of Symptoms of Dry Eye

(76) Inventors: Richard I. Blackwell, Webster, NY (US); Jay F. Kunzler, Canadaigua, NY (US); Daniel M. Ammon, JR., Webster, NY (US); Joseph C. Salamone, San Antonio, TX (US)

Correspondence Address: Bausch & Lomb Incorporated, One Bausch & Lomb Place, Rochester, NY, 14604-2701, US

(21) Appl. No.: 12/266,646

(22) Filed: Nov. 7, 2008

Related U.S. Application Data
(60) Provisional application No. 60/991,785, filed on Dec. 3, 2007.

(51) Int. Cl. A61K 31/74; A61K 31/715; A61P 27/02; A61K 31/79; A61K 31/785; A61K 31/765

(52) U.S. Cl. 424/78.04; 514/54

(57) ABSTRACT

A method for the mitigation of symptoms of dry eye is disclosed. The method involves at least (a) incorporating one or more non-functionalized polymers having one or more hydrophilic moieties into an ophthalmic device that is a polymerization product of a comonomer mixture comprising: (i) a major amount of a non-silicone-containing hydrophilic monomer; and (ii) an end terminal functionalized surfactant; and (b) inserting the ophthalmic device in the eye of a patient.

### PRIORITY CLAIMS TO PRIOR APPLICATIONS

This application claims the benefit of Provisional Patent Application No. 60/991,785 filed Dec. 3, 2007 which is incorporated by reference herein.

### BACKGROUND OF THE INVENTION

1. Technical Field

The present invention generally relates to a method for the mitigation of symptoms of dry eye such as contact lens related dry eye.

2. Description of the Related Art

Dry eye, also known generically as keratoconjunctivitis sicca and dyslacrima, is a common ophthalmological disorder affecting millions of people. A patient with dry eye may experience burning, a feeling of dryness and persistent irritation. In severe cases, dry eye can seriously impair a person's vision and hence handicap the sufferer in activities such as driving. Certain diseases such as Sjogren's disease manifest dry eye symptoms. Also, as people age, the lacrimal ducts in the eye may produce less moisture, resulting in eyes that become dry, inflamed, itchy and gritty.

Although it appears that dry eye may result from a variety of underlying, unrelated pathogenic causes, all presentations of the condition share a common effect, namely, the breakdown of the pre-ocular tear film, which commonly results in dehydration of the exposed outer surface and hence the symptoms described above.

A number of approaches exist for the treatment of dry eye. One common approach has been to supplement the ocular tear film with artificial tears throughout the day. Examples of the tear substitute approach include the use of buffered, isotonic saline solutions and aqueous solutions containing water-soluble polymers that render the solutions more viscous and thus less easily shed by the eye by the washing action of the tear fluid. See, for example, U.S. Pat. Nos. 4,409,205; 5,209,927 and 5,294,607.

Although these approaches have met with some success in certain cases, significant challenges in the treatment of dry eye nevertheless remain. Problems include the fact that the use of tear substitutes, while temporarily effective, generally requires repeated application over the course of a patient's waking hours. For example, the tear substitutes can be applied ten to twenty times over the course of a day. Such an approach is not only inconvenient and time consuming, but not very effective in preventing at least the initiation of dry-eye symptoms. Although increasing the viscosity of the dry-eye product may extend the product's duration in the eye to a limited extent, still further increases in duration would be highly desirable.

Soft contact lenses have been available since the 1980s. While there are many people who can successfully wear contact lenses, there are a number of people who can wear contact lenses for only short periods of time due to contact lens related dry eye. Symptoms of this disorder include thin and/or unstable tear films, corneal staining and subjective symptoms such as ocular discomfort, burning/stinging and dryness. Contact lens wear may trigger the onset of these symptoms or may exacerbate the symptoms. People with contact lens related dry eye generally can comfortably wear contact lenses only for limited periods of time (e.g., less than 6 hours and in some cases less than four hours).

U.S. Patent Application Publication No. 20070043140 (“the '140 application”) discloses a method for the mitigation of symptoms of contact lens related dry eye involving contacting the surface of the eye of a patient in need of mitigation of symptoms for contact lens related dry eye with a contact lens comprising senofilcon A. The '140 application further discloses that a lubricious polymer such as poly-N-vinyl pyrrolidone can be incorporated into the lens polymer without chemical bonding, copolymerized into the lens matrix or coated onto the contact lens. The contact lenses disclosed in the '140 application may have water contents of at least about 30%, and preferably between about 30 and about 50%.

U.S. Pat. No. 7,037,469 (“the '469 patent”) and U.S. Pat. No. 7,247,270 (“the '270 patent”) disclose a method of reducing swelling in a hydrogel contact lens involving contacting a hydrogel contact lens with a multi-purpose solution containing one or more polyethers such as a combination of poloxamer 407 and poloxamine 1107 in an amount ranging from about 2 wt. % to about 5 wt. % and polyquaternium-10 to absorb the solution into the hydrogel contact lens; and placing the contact lens into the eye, wherein the solution is released over a period of time from the contact lens and prevents swelling of the contact lens over the period of time. The '469 and '270 patents further disclose that the solution further contains a buffer, a tonicity adjusting agent and water soluble viscosity builders.

Accordingly, there remains a need for methods for mitigating symptoms of dry eye.

### SUMMARY OF THE INVENTION

In accordance with one embodiment of the present invention, a method for the mitigation of symptoms of dry eye is provided comprising (a) incorporating one or more non-functionalized polymers having one or more hydrophilic moieties into an ophthalmic device that is a polymerization product of a comonomer mixture comprising: (i) a major amount of a non-silicone-containing hydrophilic monomer; and (ii) an end terminal functionalized surfactant; and (b) inserting the ophthalmic device in the eye of a patient.

In accordance with a second embodiment of the present invention, a method for the mitigation of symptoms of dry eye is provided comprising (a) incorporating one or more non-functionalized polymers having one or more hydrophilic moieties into an ophthalmic device that is a polymerization product of a comonomer mixture comprising: (i) a major amount of a non-silicone-containing hydrophilic monomer; (ii) a hydrophobic monomer; and (iii) an end terminal functionalized surfactant, wherein the ophthalmic device has an equilibrium water content of at least about 70 weight percent; and (b) inserting the ophthalmic device in the eye of a patient.

In accordance with a third embodiment of the present invention, a method for the mitigation of symptoms of dry eye is provided comprising (a) providing an ophthalmic device that is a polymerization product of a comonomer mixture comprising: (i) a major amount of a non-silicone-containing hydrophilic monomer; and (ii) an end terminal functionalized surfactant; (b) contacting the ophthalmic device with a solution comprising one or more non-functionalized polymers having one or more hydrophilic moieties for a time period sufficient to incorporate the one or more non-functionalized polymers having one or more hydrophilic moieties into the ophthalmic device; and (c) inserting the ophthalmic device in the eye of a patient.

By incorporating one or more non-functionalized polymers having one or more hydrophilic moieties into an ophthalmic device that is a polymerization product of a comonomer mixture comprising: (i) a major amount of a non-silicone-containing hydrophilic monomer; and (ii) an end terminal functionalized surfactant, the non-functionalized polymer(s) will migrate to the surface of the lens in a sustained release manner thereby mitigating the systems of dry eye for a sustained period of time as compared to an ophthalmic device that is formed from a polymerization product of a comonomer mixture which does not contain an end terminal functionalized surfactant.

### BRIEF DESCRIPTION OF DRAWINGS

FIG. 1 is a bar graph showing the release rates of Pluronic® F38 from the ophthalmic devices of Examples 3-5 over a period of 0 to 4 hours.

FIG. 2 is a bar graph showing the release rates of Pluronic® F38 from the ophthalmic devices of Examples 3-5 over a period of 5 to 8 hours.

FIG. 3 is a bar graph showing the release rates of Pluronic® F38 from the ophthalmic device of Example 6 over a period of 0 to 4 hours.

FIG. 4 is a bar graph showing the release rates of Pluronic® F38 from the ophthalmic device of Example 6 over a period of 5 to 8 hours.

FIG. 5 is a bar graph showing the release rates of Pluronic® F127 from the ophthalmic devices of Examples 7 and 8 over a period of 0 to 4 hours.

FIG. 6 is a bar graph showing the release rates of Pluronic® F127 from the ophthalmic devices of Examples 7 and 8 over a period of 5 to 8 hours.

FIG. 7 is a bar graph showing the release rates of Pluronic® F127 from the ophthalmic devices of Comparative Examples A-C over a period of 0 to 4 hours.

FIG. 8 is a bar graph showing the release rates of Pluronic® F127 from the ophthalmic devices of Comparative Examples A-C over a period of 5 to 8 hours.

FIG. 9 is a bar graph showing the release rates of Pluronic® F127 from the ophthalmic devices of Comparative Examples D-G over a period of 0 to 4 hours.

FIG. 10 is a bar graph showing the release rates of Pluronic® F127 from the ophthalmic devices of Comparative Examples D-G over a period of 5 to 8 hours.

### DETAILED DESCRIPTION OF THE PREFERRED EMBODIMENTS

The present invention is directed to methods for mitigating symptoms of dry eye such as contact lens dry eye. In general, the methods involve at least (a) incorporating one or more non-functionalized polymers having one or more hydrophilic moieties into a high water content ophthalmic device; and (b) inserting the ophthalmic device in the eye of a patient. As used herein, the terms “opthalmic device” and “lens” refer to devices that reside in or on the eye. These devices can provide optical correction, wound care, drug delivery, diagnostic functionality or cosmetic enhancement or any combination of these properties. Representative examples of such devices include, but are not limited to, soft contact lenses, e.g., soft, hydrogel lens, soft, non-hydrogel lens and the like, hard contact lenses, e.g., hard, gas permeable lens materials and the like, intraocular lenses, overlay lenses, ocular inserts, optical inserts and the like. As is understood by one skilled in the art, a lens is considered to be “soft” if it can be folded back upon itself without breaking. The ophthalmic devices such as contact lenses of the present invention can be spherical, toric, bifocal, may contain cosmetic tints, opaque cosmetic patterns, combinations thereof and the like.

The high water content ophthalmic devices for use in the methods of the present invention are a polymerization product of a comonomer mixture containing at least (a) a major amount of a non-silicone-containing hydrophilic monomer; and (b) an end terminal functionalized surfactant. Although the invention is applicable to a variety of high water content ophthalmic devices, the invention is especially useful and advantageous for high water content contact lenses. The high water content ophthalmic devices used herein have an equilibrium water content of at least about 70 weight percent and preferably at least about 80 weight percent.

Suitable non-silicone-containing hydrophilic monomers include amides such as N,N-dimethylacrylamide and N,N-dimethylmethacrylamide; cyclic lactams such as N-vinyl-2-pyrrolidone; poly(alkene glycols) functionalized with polymerizable groups and the like and mixtures thereof. Examples of useful functionalized poly(alkene glycols) include poly(diethylene glycols) of varying chain length containing monomethacrylate or dimethacrylate end caps. In a preferred embodiment, the poly(alkene glycol) polymer contains at least two alkene glycol monomeric units. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art.

The hydrophilic monomers such as a N-vinyl lactam-containing monomer are present in the comonomer mixture in a major amount, e.g., an amount greater than or equal to about 70 weight percent and preferably greater than or equal to about 80 weight percent, based on the total weight of the comonomer mixture.

A suitable end terminal functionalized surfactant includes, by way of example, one or more end terminal functionalized polyethers. Useful polyethers to be end terminal functionalized comprise one or more chains or polymeric components which have one or more (—O—R—) repeats units wherein R is an alkylene or arylene group having 2 to about 6 carbon atoms. The polyethers may be derived from block copolymers formed from different ratio components of ethylene oxide (EO) and propylene oxide (PO). Such polyethers and their respective component segments may include different attached hydrophobic and hydrophilic chemical functional group moieties and segments.

A representative example of a suitable polyether which can be end terminal functionalized is a poloxamer block copolymer. One specific class of poloxamer block copolymers are those available under the trademark Pluronic (BASF Wyandotte Corp., Wyandotte, Mich.). Poloxamers include Pluronics and reverse Pluronics. Pluronics are a series of ABA block copolymers composed of poly(ethylene oxide)-poly(propylene oxide)-poly(ethylene oxide) blocks as generally represented in Figure I:

HO(C₂H₄O)ₐ(C₃H₆O)_{b}(C₂H₄O)ₐH (I)

wherein a is independently at least 1 and b is at least 1.

Reverse Pluronics are a series of BAB block copolymers, respectively composed of poly(propylene oxide)-poly(ethylene oxide)-poly(propylene oxide) blocks as generally represented in Figure II:

HO(C₂H₄O)_{b}(C₃H₆O)ₐ(C₂H₄O)_{b}H (II)

wherein a is at least 1 and b is independently at least 1. The poly(ethylene oxide), PEO, blocks are hydrophilic, whereas the poly(propylene oxide), PPO, blocks are hydrophobic in nature. The poloxamers in each series have varying ratios of PEO and PPO which ultimately determines the hydrophilic-lipophilic balance (HLB) of the material, i.e., the varying HLB values are based upon the varying values of a and b, a representing the number of hydrophilic poly(ethylene oxide) units (PEO) being present in the molecule and b representing the number of hydrophobic poly(propylene oxide) units (PPO) being present in the molecule.

Poloxamers and reverse poloxamers have terminal hydroxyl groups that can be terminal functionalized. An example of a terminal functionalized poloxamer and as discussed hereinbelow is poloxamer dimethacrylate (e.g., Pluronic® F127 dimethacrylate) as disclosed in U.S. Patent Application Publication No. 2003/0044468. Other examples include glycidyl-terminated copolymers of polyethylene glycol and polypropylene glycol as disclosed in U.S. Pat. No. 6,517,933.

Another example of a suitable polyether which can be end terminal functionalized is a poloxamine block copolymer. While the poloxamers and reverse poloxamers are considered to be difunctional molecules (based on the terminal hydroxyl groups), the poloxamines are in a tetrafunctional form, i.e., the molecules are tetrafunctional block copolymers terminating in primary hydroxyl groups and linked by a central diamine. One specific class of poloxamine block copolymers are those available under the trademark Tetronic (BASF). Poloxamines include Tetronic and reverse Tetronics. Poloxamines have the following general structure of Formula III:

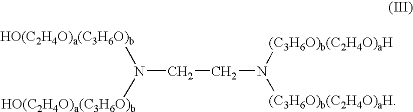

wherein a is independently at least 1 and b is independently at least 1.

The poloxamer and/or poloxamine is functionalized to provide the desired reactivity at the end terminal of the molecule. The functionality can be varied and is determined based upon the intended use of the functionalized PEO- and PPO-containing block copolymers. That is, the PEO- and PPO-containing block copolymers are reacted to provide end terminal functionality that is complementary with the intended device forming monomer mixture. The term block copolymer as used herein shall be understood to mean a poloxamer and/or poloxamine as having two or more blocks in their polymeric backbone(s).

Generally, selection of the functional end group is determined by the functional group of the reactive molecule(s) in the monomer mix. For example, if the reactive molecule contains a carboxylic acid group, glycidyl methacrylate can provide a methacrylate end group. If the reactive molecule contains hydroxy or amino functionality, isocyanato ethyl methacrylate or (meth)acryloyl chloride can provide a methacrylate end group and vinyl chloro formate can provide a vinyl end group. A wide variety of suitable combinations of ethylenically unsaturated end groups and reactive molecules will be apparent to those of ordinary skill in the art. For example, the functional group may comprise a moiety selected from amine, hydrazine, hydrazide, thiol (nucleophilic groups), carboxylic acid, carboxylic ester, including imide ester, orthoester, carbonate, isocyanate, isothiocyanate, aldehyde, ketone, thione, alkenyl, acrylate, methacrylate, acrylamide, sulfone, maleimide, disulfide, iodo, epoxy, sulfonate, thiosulfonate, silane, alkoxysilane, halosilane, and phosphoramidate. More specific examples of these groups include succinimidyl ester or carbonate, imidazolyl ester or carbonate, benzotriazole ester or carbonate, p-nitrophenyl carbonate, vinyl sulfone, chloroethylsulfone, vinylpyridine, pyridyl disulfide, iodoacetamide, glyoxal, dione, mesylate, tosylate, and tresylate. Also included are other activated carboxylic acid derivatives, as well as hydrates or protected derivatives of any of the above moieties (e.g. aldehyde hydrate, hemiacetal, acetal, ketone hydrate, hemiketal, ketal, thioketal, thioacetal). Preferred electrophilic groups include succinimidyl carbonate, succinimidyl ester, maleimide, benzotriazole carbonate, glycidyl ether, imidazoyl ester, p-nitrophenyl carbonate, acrylate, tresylate, aldehyde, and orthopyridyl disulfide.

Representative examples of reaction sequences by which PEO- and PPO-containing block copolymers can be end-functionalized are provided below.

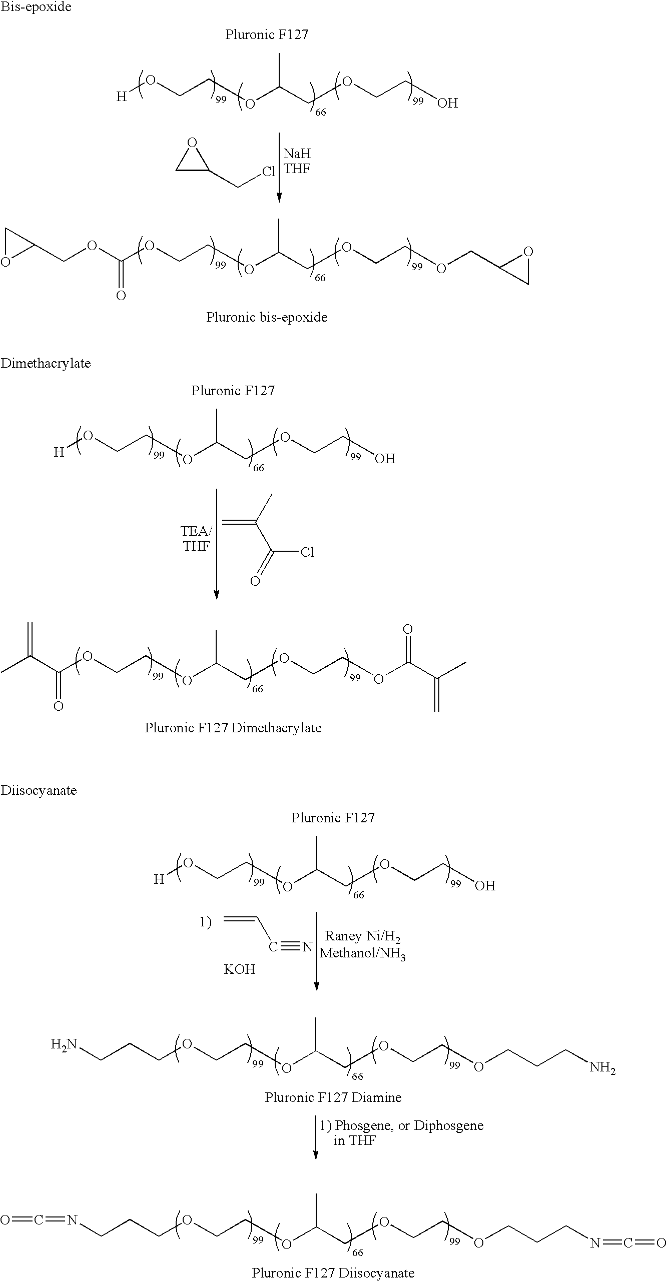

Other exemplary, but non-limiting, examples of reactions for providing functionalized termini for PEO- and PPO-containing block copolymers. It is to be understood that one of ordinary skill in the art would be able to determine other reaction methods without engaging in an undue amount of experimentation. It should also be understood that any particular block copolymer molecule shown is only one chain length of a polydispersed population of the referenced material.

PEO- and PPO-containing block copolymers are presently preferred. An example of such a copolymer that can be used with the methods of the present invention is Pluronic® F127, a block copolymer having the structure [(polyethylene oxide)₉₉-(polypropylene oxide)₆₆-(polyethylene oxide)₉₉]. The terminal hydroxyl groups of the copolymer are functionalized to allow for the reaction of the copolymer with other ophthalmic device forming monomers.

In one embodiment, an end terminal functionalized surfactant is selected from the group consisting of poloxamers having at least one end terminal functionalized, reverse poloxamers having at least one end terminal functionalized, poloxamines having at least one end terminal functionalized, reverse poloxamines having at least one end terminal functionalized and mixtures thereof.

It is particularly advantageous to employ an end terminal functionalized surfactant that possesses a relatively similar structure as the non-functionalized polymers having one or more hydrophilic moieties as discussed hereinbelow. For example, the non-functionalized polymers having one or more hydrophilic moieties can be the same poloxamer as the end terminal functionalized surfactants except the poloxamer of the end terminal functionalized surfactants is end terminated with a functional group as discussed above. The incorporation of relatively small amounts of the end terminal functionalized surfactants has been shown to affect the release of the non-functionalized polymer(s) having hydrophilic moieties. Generally, the end terminal functionalized surfactants will be present in the comonomer mixtures in an amount ranging from about 0.01 to about 20 weight percent, preferably from about 1 to about 10 weight percent, and most preferably from about 3 to about 6 weight percent, based on the total weight of the comonomer mixture.

The comonomer mixture can further contain one or more hydrophobic monomers. Suitable hydrophobic monomers include ethylenically unsaturated hydrophobic monomers such as, for example, (meth)acrylate-containing hydrophobic monomers, N-alkyl(meth)acrylamide-containing hydrophobic monomers, alkyl vinylcarbonate-containing hydrophobic monomers, alkyl vinylcarbamate-containing hydrophobic monomers, fluoroalkyl(meth)acrylate-containing hydrophobic monomers, N-fluoroalkyl(meth)acrylamide-containing hydrophobic monomers, N-fluoroalkyl vinylcarbonate-containing hydrophobic monomers, N-fluoroalkyl vinylcarbamate-containing hydrophobic monomers, silicone-containing (meth)acrylate-containing hydrophobic monomers, (meth)acrylamide-containing hydrophobic monomers, vinyl carbonate-containing hydrophobic monomers, vinyl carbamate-containing hydrophobic monomers, styrenic-containing hydrophobic monomers, polyoxypropylene(meth)acrylate-containing hydrophobic monomers and the like and mixtures thereof. As used herein, the term “(meth)” denotes an optional methyl substituent. Thus, terms such as “(meth)acrylate” denotes either methacrylate or acrylate, and “(meth)acrylamide” denotes either methacrylamide or acrylamide.

In one embodiment, a preferred hydrophobic monomer is represented by Formula IV:

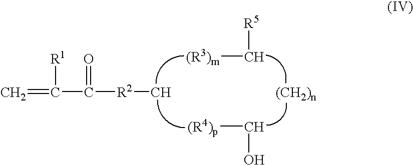

wherein R¹ is methyl or hydrogen; R² is —O— or —NH—; R³ and R⁴ are independently a divalent radical selected from the group consisting of —CH₂—, —CHOH— and —CHR⁶—; R⁵ and R⁶ are independently a branched C₃-C₈ alkyl group; and n is an integer of at least 1, and m and p are independently 0 or an integer of at least 1, provided that the sum of m, p and n is 2, 3, 4 or 5. Representative examples of hydrophobic monomers (b) include, but are not limited to, 4-t-butyl-2-hydroxycyclohexyl methacrylate (TBE); 4-t-butyl-2-hydroxycyclopentyl methacrylate; 4-t-butyl-2-hydroxycyclohexyl methacrylamide (TBA); 6-isopentyl-3-hydroxycyclohexyl methacrylate; and 2-isohexyl-5-hydroxycyclopentyl methacrylamide. Preferred hydrophobic monomers include compounds of Formula IV wherein R³ is —CH₂—, m is 1 or 2, p is 0, and the sum of m and n is 3 or 4. TBE and TBA are especially preferred.

The hydrophobic monomer will ordinarily be present in the comonomer mixture in an amount ranging from about 0.5 to about 25 weight percent and preferably from about 1 to about 10 weight percent, based on the total weight of the comonomer mixture.

Suitable crosslinking agents for use herein are known in the art. A useful crosslinking monomer can have at least two polymerizable functional groups. Representative crosslinking agents include, but are not limited to, allyl methacrylate and ethylene glycol dimethyacrylate (EGDMA). The crosslinking agent is generally used in amounts of less than about 5 weight percent, and generally less than about 2 weight percent, based on the total weight of the comonomer mixture.

The comonomer mixture may further contain, as necessary and within limits not to impair the purpose and effect of the present invention, various additives such as antioxidant, coloring agent, ultraviolet absorber, lubricant internal wetting agents, toughening agents and the like and other constituents as is well known in the art.

The polymerization products disclosed herein can be obtained by polymerizing the comonomer mixture containing at least (a) a major amount of a non-silicone-containing hydrophilic monomer; and (b) an end terminal functionalized surfactant by conventional techniques for polymerization, typically thermal or photochemical polymerization. For thermal polymerization, a temperature from about 40° C. to about 120° C. is used. For photochemical polymerization, radiation such as gamma, ultraviolet (UV) light, visible, or microwave radiation may be used.

Polymerization can be performed in a reaction medium, such as, for example, a solution or dispersion using a solvent, e.g., water or an alkanol containing from 1 to 4 carbon atoms such as methanol, ethanol or propan-2-ol. Alternatively, a mixture of any of the above solvents may be used.

A polymerization initiator may be included in the mixture to facilitate the polymerization step. Representative examples of free radical thermal polymerization initiators include organic peroxides such as, for example, acetal peroxide, lauroyl peroxide, decanoyl peroxide, stearoyl peroxide, benzoyl peroxide, tertiarylbutyl peroxypivalate, peroxydicarbonate, and the like and mixtures thereof. Representative examples of UV initiators include those known in the field such as, for example, benzoin methyl ether, benzoin ethyl ether, Darocure 1173, 1164, 2273, 1116, 2959, 3331 (EM Industries) and Igracure 651 and 184 (Ciba-Geigy), and the like and mixtures thereof. Generally, the initiator will be employed in the comonomer mixture at a concentration at about 0.1 to about 5 percent by weight of the total mixture.

Generally, polymerization can be carried out for about 15 minutes to about 72 hours and under an inert atmosphere of, for example, nitrogen or argon. If desired, the resulting polymerization product can be dried under vacuum, e.g., for about 5 to about 72 hours.

The polymerization products can be formed into ophthalmic devices by, for example, spincasting processes (e.g., those disclosed in U.S. Pat. Nos. 3,408,429 and 3,496,254), cast molding, lathe cutting, or any other known method for making the devices. Polymerization may be conducted either in a spinning mold, or a stationary mold corresponding to a desired shape. The ophthalmic device may be further subjected to mechanical finishing, as occasion demands. Polymerization may also be conducted in an appropriate mold or vessel to form buttons, plates or rods, which may then be processed (e.g., cut or polished via lathe or laser) to provide an ophthalmic device having a desired shape.

Next, one or more non-functionalized polymers having one or more hydrophilic moieties are incorporated into the foregoing ophthalmic devices in an amount sufficient to mitigate the symptoms of dry eye. Suitable non-functionalized polymers having hydrophilic moieties include non-functionalized polyethers, non-functionalized polysaccharides, non-functionalized polyamides, non-functionalized polylactones, non-functionalized polyalcohols, non-functionalized polyimides, non-functionalized polylactams, copolymers thereof and the like and mixtures thereof.

Suitable non-functionalized polyethers may be derived from block copolymers formed from different ratio components of ethylene oxide (EO) and propylene oxide (PO). Examples of a suitable polyether include the poloxamer block copolymers available under the trademark Pluronic (BASF Wyandotte Corp., Wyandotte, Mich.) and include Pluronics and reverse Pluronics as discussed above. In one embodiment, a block copolymer that can be used herein is Pluronic® F127, a block copolymer having the structure [(polyethylene oxide)₉₉-(polypropylene oxide)₆₆-(polyethylene oxide)₉₉]. In another embodiment, a non-functionalized polyether is Pluronic® F38.

Another example of a suitable non-functionalized polyether is a poloxamine block copolymer available under the trademark Tetronic (BASF) and include Tetronic and reverse Tetronics as discussed above.

Suitable non-functionalized polysaccharides are derived from the families based on cellulosics, guar (e.g., hydroxypropyl guar), starch, dextran, chitosan, locust bean gum, gum tragacanth, curdlan, pullulan and seleroglucan. Representative examples of cellulose polymers include hydroxypropyl cellulose, hydroxypropyl methyl cellulose, hydroxyethyl cellulose, carboxy methylcellulose, methyl cellulose and the like and mixtures thereof.

Representative examples of other non-functionalized polymers include, but are not limited to, poly-N-vinyl pyrrolidone, poly(N-vinyl-N-methylacetamide), poly-N-vinyl-2-piperidone, poly-N-vinyl-2-caprolactam, poly-N-vinyl-3-methyl-2-caprolactam, poly-N-vinyl-3-methyl-2-piperidone, poly-N-vinyl-4-methyl-2-piperidone, poly-N-vinyl-4-methyl-2-caprolactam, poly-N-vinyl-3-ethyl-2-pyrrolidone, and poly-N-vinyl-4,5-dimethyl-2-pyrrolidone, polyvinylimidazole, poly-N-N-dimethylacrylamide, polyvinyl alcohol, polyethylene oxide, poly 2 ethyl oxazoline, heparin polysaccharides, mixtures and copolymers (including block or random, branched, multichain, comb-shaped or star shaped) thereof.

In one embodiment, a solution containing at least the non-functionalized polymer(s) having one or more hydrophilic moieties is an aqueous solution containing the non-functionalized polymer(s) with water. In another embodiment, the solution may be formulated as a “multi-purpose solution”. A multi-purpose solution is useful for cleaning, disinfecting, storing, and rinsing a lens, particularly soft contact lenses. Multi-purpose solutions do not exclude the possibility that some wearers, for example, wearers particularly sensitive to chemical disinfectants or other chemical agents, may prefer to rinse or wet a contact lens with another solution, for example, a sterile saline solution prior to insertion of the lens. The term “multi-purpose solution” also does not exclude the possibility of periodic cleaners not used on a daily basis or supplemental cleaners for further removing proteins, for example, enzyme cleaners, which are typically used on a weekly basis. By the term “cleaning” is meant that the solution contains one or more agents in sufficient concentrations to loosen and remove loosely held lens deposits and other contaminants on the surface of a contact lens, which may be used in conjunction with digital manipulation (e.g., manual rubbing of the lens with a solution) or with an accessory device that agitates the solution in contact with the lens, for example, a mechanical cleaning aid.

Traditionally, multi-purpose solutions on the market have required a regimen involving mechanical rubbing of the lens with the multi-purpose solution, in order to provide the required disinfection and cleaning. Such a regimen is required under governmental regulatory authorities (e.g., the FDA or U.S. Food & Drug Administration (FDA)) for a Chemical Disinfection System that does not qualify as a Chemical Disinfecting Solution. In one embodiment of the present invention, it is possible to formulate a solution that, on one hand, is able to provide improved cleaning and disinfection in the absence of a rubbing regimen and, on the other hand, is gentle enough to be used as a wetting agent, e.g. as an eye drop. For example, a product qualifying as a Chemical Disinfecting Solution must meet biocidal performance criteria established by the US FDA for Contact Lens Care Products (May 1, 1997) which criteria does not involve rubbing of the lenses. In one embodiment of the present invention, a composition is formulated to meet the requirements of the FDA or ISO Stand-Alone Procedure for contact lens disinfecting products. Similarly, the compositions of the present invention can be formulated to provide enhanced cleaning without the use of a rubbing regimen. Such formulations may ensure higher patient compliance and greater universal appeal than traditional multi-purpose disinfecting and cleaning products. A multi-purpose solution preferably has a viscosity of less than about 75 cps, preferably about 1 to about 50 cps, and most preferably about 1 to about 25 cps and is preferably at least about 95 percent weight by volume water in the total composition.

The aqueous ophthalmic solutions of this embodiment may contain, in addition to the copolymers described above, one or more antimicrobial agents, preservatives and the like. The compositions generally include a primary antimicrobial agent. Antimicrobial agents suitable for use in the present invention include chemicals that derive their antimicrobial activity through a chemical or physiochemical interaction with the microbial organisms. These agents may be used alone or in combination.

Suitable known ophthalmically acceptable antimicrobial agents include, but are not limited to, a biguanide or a salt or free base thereof, quaternary ammonium compound or a salt thereof or free base thereof; terpene or derivative thereof, a branched, glycerol monoalkyl ether, a branched, glycerol monoalkyl amine, a branched, glycerol monoalkyl sulphide, a fatty acid monoester, wherein the fatty acid monoester comprises an aliphatic fatty acid portion having six to fourteen carbon atoms, and an aliphatic hydroxyl portion, amidoamine compound, and the like and combinations thereof.

Suitable biguanide antimicrobial agents for use in the ophthalmic compositions of the present inventions can be any biguanide or salt thereof known in the art. Representative biguanides include non-polymeric biguanides, polymeric biguanides, salts thereof, free bases thereof and the like and mixtures thereof. Representative non-polymeric biguanides are the bis(biguanides), such as alexidine, chlorhexidine, salts of alexidine, e.g., alexidine HCl, salts of chlorhexidine, alexidine free base, and the like and mixtures thereof. The salts of alexidine and chlorhexidine can be either organic or inorganic and are typically disinfecting nitrates, acetates, phosphates, sulfates, halides and the like.

Representative polymeric biguanides include polymeric hexamethylene biguanides (PHMB) (commercially available from Zeneca, Wilmington, Del.), their polymers and water-soluble salts. In one embodiment, water-soluble polymeric biguanides for use herein can have a number average molecular weight of at least about 1,000 and more preferably a number average molecular weights from about 1,000 to about 50,000. Suitable water-soluble salts of the free bases include, but are not limited to, hydrochloride, borate, acetate, gluconate, sulfonate, tartrate and citrate salts. Generally, the hexamethylene biguanide polymers, also referred to as polyaminopropyl biguanide (PAPB), have number average molecular weights of up to about 100,000. Such compounds are known and are disclosed in U.S. Pat. No. 4,758,595 which patent is incorporated herein be reference.

PHMB is best described as a polymeric biguanide composition comprising at least three and preferably at least six biguanide polymers, which we refer to as PHMB-A, PHMB-CG and PHMB-CGA, the general chemical structures of which are depicted below.

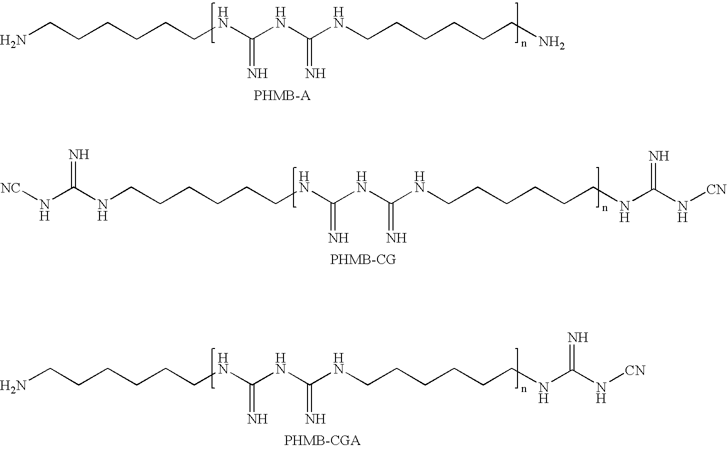

For each of these polymers, “n” represents the average number of repeating groups. Actually, a distribution of polymer length would exist for each of the polymers shown. The prior synthetic routes to PHMB provided a polymeric biguanide composition with about 50% by weight of the polymeric composition as PHMB-CGA, that is, having a cyanoguanidino end cap on one end and an amine on the other end, about 25% by weight PHMB-A and about 25% by weight PHMB-CG. Given this approximate weight ratio of the three major PHMB polymers above, the percentage of cyanoguardino end caps is also about 50% of the total number of terminal groups. In this application we refer to this conventional polymeric biguanide composition as poly(hexamethylene biguanide) or PHMB.

A new synthetic route to polymeric biguanide compositions is described in copending U.S. provisional application Ser. No. 60/853,579, filed Oct. 23, 2006, and 60/895,770, filed Mar. 20, 2007, the entire disclosure of each of which is incorporated by reference herein. The new synthetic route provides a polymeric biguanide composition comprising less than 18 mole % of terminal amine groups as measured by ¹³CNMR. The polymeric biguanide composition can also be characterized by a relative increase in the molar concentration of terminal guanidine groups or terminal cyanoguardino groups. For example, in one embodiment, the biguanide composition comprises less than about 18 mole % of terminal amine groups and about 40 mol % or greater of terminal guanidine groups. In another embodiment, the biguanide composition comprises less than about 18 mole % of terminal amine groups and about 55 mol % or greater of terminal guanidine groups.

In this application, we refer to this biguanide composition as PHMB-CG*. We also refer to polymeric biguanide compositions in the generic sense as “hexamethylene biguanides”, which one of ordinary skill in the art would recognize to include both PHMB as well as PHMB-CG*.

Representative examples of suitable quaternary ammonium compounds for use in the ophthalmic compositions of the present invention include, but are not limited to, poly[(dimethyliminio)-2-butene-1,4-diyl chloride] and [4-tris(2-hydroxyethyl)ammonio]-2-butenyl-w-[tris(2-hydroxyethyl)ammonio]-dichloride (chemical registry no. 75345-27-6) generally available as Polyquatemium 1 under the tradename Onamer® M (Stepan Company, Northfield, Ill.), and the like and mixtures thereof.

Suitable terpene antimicrobial agents for use in the ophthalmic compositions of the present invention include any monoterpene, sesquiterpene and/or diterpene or derivatives thereof. Acyclic, monocyclic and/or bicyclic mono-, sesqui- and/or diterpenes, and those with higher numbers of rings, can be used. A “derivative” of a terpene as used herein shall be understood to mean a terpene hydrocarbon having one or more functional groups such as terpene alcohols, terpene ethers, terpene esters, terpene aldehydes, terpene ketones and the like and combinations thereof. Here, both the trans and also the cis isomers are suitable. The terpenes as well as the terpene moiety in the derivative can contain from 6 to about 100 carbon atoms and preferably from about 10 to about 25 carbon atoms.

Representative examples of suitable terpene alcohol antimicrobial agents include verbenol, transpinocarveol, cis-2-pinanol, nopol, isoborneol, carbeol, piperitol, thymol, α-terpineiol, terpinen-4-ol, menthol, 1,8-terpin, dihydro-terpineol, nerol, geraniol, linalool, citronellol, hydroxycitronellol, 3,7-dimethyl octanol, dihydro-myrcenol, tetrahydro-alloocimenol, perillalcohol, falcarindiol and the like and mixtures thereof.

Representative examples of suitable terpene ether and terpene ester antimicrobial agents include 1,8-cineole, 1,4-cineole, isobornyl methylether, rose pyran, α-terpinyl methyl ether, menthofuran, trans-anethole, methyl chavicol, allocimene diepoxide, limonene mono-epoxide, isobornyl acetate, nonyl acetate, α-terpinyl acetate, linalyl acetate, geranyl acetate, citronellyl acetate, dihydro-terpinyl acetate, meryl acetate and the like and mixtures thereof.

Representative examples of terpene aldehyde and terpene ketone antimicrobial agents include myrtenal, campholenic aldehyde, perillaldehyde, citronellal, citral, hydroxy citronellal, camphor, verbenone, carvenone, dihydro-carvone, carvone, piperitone, menthone, geranyl acetone, pseudo-ionone, α-ionine, iso-pseudo-methyl ionone, n-pseudo-methyl ionone, iso-methyl ionone, n-methyl ionone and the like and mixtures thereof. Any other terpene hydrocarbons having functional groups known in the art may be used herein.

In one embodiment, suitable terpenes or derivatives thereof as antimicrobial agents include, but are not limited to, tricyclene, α-pinene, terpinolene, carveol, amyl alcohol, nerol, β-santalol, citral, pinene, nerol, b-ionone, caryophillen (from cloves), guaiol, anisaldehyde, cedrol, linalool, d-limonene (orange oil, lemon oil), longifolene, anisyl alcohol, patchouli alcohol, α-cadinene, 1,8-cineole, ρ-cymene, 3-carene, ρ-8-mentane, trans-menthone, borneol, α-fenchol, isoamyl acetate, terpin, cinnamic aldehyde, ionone, geraniol (from roses and other flowers), myrcene (from bayberry wax, oil of bay and verbena), nerol, citronellol, carvacrol, eugenol, carvone, α-terpineol, anethole, camphor, menthol, limonene, nerolidol, farnesol, phytol, carotene (vitamin A₁), squalene, thymol, tocotrienol, perillyl alcohol, borneol, simene, carene, terpenene, linalool, 1-terpene-4-ol, zingiberene (from ginger) and the like and mixtures thereof.

In one embodiment, the compound of component (ii) of the ophthalmic composition comprises a branched, glycerol monoalkyl ether. In another embodiment, the compound of component (ii) of the ophthalmic composition comprises a branched, glycerol monoalkyl amine. In another embodiment, the compound of component (ii) of the ophthalmic composition comprises a branched, glycerol monoalkyl sulphide. In still another embodiment, the compound of component (ii) of the ophthalmic composition comprises any one mixture of a branched, glycerol monoalkyl ether, a branched, glycerol monoalkyl amine or a branched, glycerol monoalkyl sulphide.

In one embodiment, the branched, glycerol monoalkyl ether for use in the ophthalmic compositions of the present invention is 3-[(2-ethylhexyl)oxy]-1,2-propanediol (EHOPD). In another embodiment, the branched, glycerol monoalkyl amine is 3-[(2-ethylhexyl)amino]-1,2-propanediol (EHAPD). In another embodiment, the branched, glycerol monoalkyl sulphide is 3-[(2-ethylhexyl)thio]-1,2-propanediol (EHSPD). In still another embodiment, the ophthalmic composition comprises any one mixture of EHOPD, EHAPD and EHSPD. The chemical structures of EHOPD, EHAPD and EHSPD are provided below.

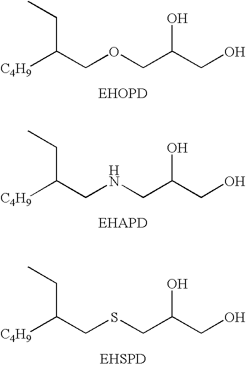

EHOPD is also referred to as octoxyglycerin and is sold under the tradename Sensiva® SC50 (Schülke & Mayr). EHOPD is a branched, glycerol monoalkyl ether known to be gentle to the skin, and to exhibit antimicrobial activity against a variety of Gram-positive bacteria such as Micrococcus luteus, Corynebacterium aquaticum, Corynebacterium flavescens, Corynebacterium callunae, and Corynebacterium nephredi. Accordingly, EHOPD is used in various skin deodorant preparations at concentrations between about 0.2 and 3 percent by weight. EHAPD can be prepared from 2-ethylhexylamine and 2,3-epoxy-1-propanediol using chemistry well known to those of ordinary skill in the art. EHSPD can be prepared from 2-ethylhexylthiol and 2,3-epoxy-1-propanediol using chemistry well known to those of ordinary skill in the art.

Suitable fatty acid monoester for use in the ophthalmic compositions of the present invention include those fatty acid monoesters comprising an aliphatic fatty acid portion having six to fourteen carbon atoms, and an aliphatic hydroxyl portion.

The term “aliphatic” refers to a straight or branched, saturated or unsaturated hydrocarbon having six to fourteen carbon atoms. In one embodiment, the aliphatic fatty acid portion is a straight chain, saturated or unsaturated hydrocarbon with eight to ten carbons. In another embodiment, the aliphatic fatty acid portion is a branched chain, saturated or unsaturated hydrocarbon with eight to ten carbons.

The aliphatic hydroxyl portion of the fatty acid monoester can be any aliphatic compound with at least one hydroxyl group. In many of the embodiments, the aliphatic hydroxyl portion will have from three to nine carbons. The aliphatic hydroxyl portion can include, but is not limited to, propylene glycol, glycerol, a polyalkylene glycol, e.g., polyethylene glycol or polypropylene glycol, a cyclic polyol, e.g., sorbitan, glucose, mannose, sucrose, fructose, fucose and inisitol and derivatives thereof, and a linear polyol, e.g., mannitol and sorbitol and derivatives thereof and the like and mixtures thereof.

Representative examples of suitable amidoamines for use in the ophthalmic compositions of the present inventions include those amidoamines of the general formula:

R¹²—(OCH₂CH₂)ₘ—X—(CH₂)ₙ—Y

wherein R¹² is a is C₆-C₃₀ saturated or unsaturated hydrocarbon including by way of example, a straight or branched, substituted or unsubstituted alkyl, alkylaryl, or alkoxyaryl group; m is zero to 16; n is 2 to 16; X is —C(O)—NR¹³— or —R¹³N—C(O)—;Y is —N(R¹⁴)₂ wherein each of R¹³ and R¹⁴ independently are hydrogen, a C₁-C₈ saturated or unsaturated alkyl or hydroxyalkyl, or a pharmaceutically acceptable salt thereof. In one embodiment, m is 0, R¹² is heptadec-8-enyl, undecyl, undecenyl, dodecyl, tridecyl, tetradecyl, pentadecyl or heptadecyl, R² is hydrogen or methyl, and R³ is methyl or ethyl.

Some of the amidoamines utilized in the present invention are available from commercial sources. For example, myristamidopropyl dimethylamine is available from Alcon Inc. (Fort Worth, Tex.) under the tradename Aldox®; lauramidopropyl dimethylamine is available from Inolex Chemical Company (Philadelphia, Pa.) under the tradename LEXAMINE® L-13; and stearamidopropyl dimethylamine is also from Inolex Chemical Company as LEXAMINE® S-13. The above-described amidoamines can be synthesized in accordance with known techniques, including those described in U.S. Pat. No. 5,573,726.

The amount of the primary antimicrobial agent may vary depending on the specific agent employed. For the aforementioned organic nitrogen-containing agent, typically, such agents are present in concentrations ranging from about 0.00001 to about 0.5% weight percent, and more preferably, from about 0.00003% to about 0.05% weight percent. For sorbic acid, higher amounts may be required, typically about 0.01 to about 1 weight percent, more preferably about 0.1 to about 0.5 weight percent. It is preferred that the antimicrobial agent is used in an amount that will at least partially reduce the microorganism population in the formulations employed. If desired, the antimicrobial agent may be employed in a disinfecting amount, which will reduce the microbial bioburden by at least two log orders in four hours and more preferably by one log order in one hour. Most preferably, a disinfecting amount is an amount which will eliminate the microbial burden on a contact lens when used in regimen for the recommended soaking time (FDA Chemical Disinfection Efficacy Test-July, 1985 Contact Lens Solution Draft Guidelines).

The aqueous solutions of this embodiment may further contain one or more other components that are commonly present in ophthalmic solutions, for example, surfactants, tonicity adjusting agents; buffering agents; chelating agents; pH adjusting agents, viscosity modifying agents, and demulcents and the like as discussed hereinabove, and which aid in making ophthalmic compositions more comfortable to the user and/or more effective for their intended use.

The pH of the solutions and/or compositions of the present invention may be maintained within the range of pH of about 4.0 to about 9.0, preferably about 5.0 to about 8.0, more preferably about 6.0 to about 8.0, and even more preferably about 6.5 to about 7.8. In one embodiment, pH values of greater than or equal to about 7 are most preferred.

The one or more non-functionalized polymers are incorporated into the ophthalmic device, i.e., a polymer network obtained by polymerizing the foregoing comonomer mixture, by contacting the ophthalmic device with a solution containing at least the one or more of the non-functionalized polymer. The ophthalmic device is contacted with the solution for a time period sufficient to incorporate an amount of the non-functionalized polymer(s) into the device such that the non-functionalized polymer(s) is released from the ophthalmic device in a sustained manner. In one embodiment, the solution will contain the non-functionalized polymers in amounts ranging from about 0.001 to about 20 weight percent, based on the weight of the solution. In another embodiment, the solution will contain the non-functionalized polymers in amounts ranging from about 2 to about 20 weight percent, based on the weight of the solution. In yet another embodiment, the solution will contain the non-functionalized polymers in amounts ranging from about 5 to about 20 weight percent, based on the weight of the solution.

Once the non-functionalized polymer(s) has been incorporated into the ophthalmic device, the treated ophthalmic device can then be placed in the eye and worn. When the ophthalmic device is placed in the eye, the non-functionalized polymer(s) will migrate to the surface of the device and released in a sustained manner thereby mitigating the systems of dry eye for a sustained period of time while providing improved lubricity and end-of-the day comfort. If desired, the device can be removed from the eye, immersed into a new solution containing at least the non-functionalized polymer(s) and reworn.

The following examples are provided to enable one skilled in the art to practice the invention and are merely illustrative of the invention. The examples should not be read as limiting the scope of the invention as defined in the claims.

### EXAMPLE 1

Preparation of an End Terminal Functionalized Surfactant.

Pluronic® F127 (6.00 g) was placed in a round bottom flask and dried thoroughly via azeotropic distillation of toluene (100 ml). The round bottom flask was then fitted with a reflux condenser and the reaction was blanketed with nitrogen gas. Anhydrous tetrahydrofuran (THF) (60 ml) was added to the flask and the reaction was chilled to 5° C. with 15 equivalents (based upon the hydroxyl endgroups) of triethylamine (TEA) (2.0 ml) was added. Methacryoyl chloride (1.4 ml) (15 equivalents) was dropped into the reaction mixture through an addition funnel and the reaction mixture was allowed to warm to room temperature and then stirred overnight. The reaction mixture was then heated to 65° C. for 3 hours. Precipitated salt (TEA-HCl) was filtered from the reaction mixture and the filtrate was concentrated to a volume of around 355 mL and precipitated into cold heptane. Two further reprecipitations were performed to reduce the amount of TEA-HCl salt to less than 0.2% by weight. NMR analysis of the final polymer showed greater than 90% conversion of the hydroxyl endgroups to the methacrylated endgroups.

### EXAMPLE 2

Preparation of an End Terminal Functionalized Surfactant.

Pluronic® F38 (10.00 g; 2.13E-03 mol) was placed in a round bottom flask and dried thoroughly via azeotropic distillation of toluene and then dissolved in 100 mL of THF. 10 equivalents of solid NaH were added into the flask (0.51 g; 2.13E-02 mol). Next, epichlorohydrin (1.67 mL; 2.13E-03 mol) was added to the reaction mixture and mixed well. The reaction mixture was heated to reflux for 24 hours and then cooled. A scoop of magnesium sulfate and silica gel was added to the reaction mixture to remove any water, mixed well for 5 minutes and then filtered off the insolubles. The filtrate was concentrated to around 30 mL final volume and the product was precipitated into heptane and isolated by filtration. NMR confirms the presence of epoxide groups on the termini of the polymer.

### EXAMPLE 3

A monomer mixture was prepared by mixing the following components, N-vinyl-2-pyrrolidone (NVP) (90 weight percent); 4-t-butyl-2-hydroxycyclohexyl methacrylate (TBE) (10 weight percent), Pluronics® F127 dimethacrylate (DM) (HLB=22, Mw˜12600) (5 weight percent), ethylene glycol dimethacrylate (EGDMA) (0.3 weight percent) and a Vazo 64 initiator (0.5 weight percent). The monomeric mixture was cast in a polypropylene contact lens mold and thermally cured for about 4 hours. The resulting contact lens had an equilibrium water content (EWC) of approximately 82%, as calculated from the following equation:

$$( \frac{( Wet weight ( mg ) - Dry weight ( mg ) )}{Wet weight ( mg )} ) \times 100$$

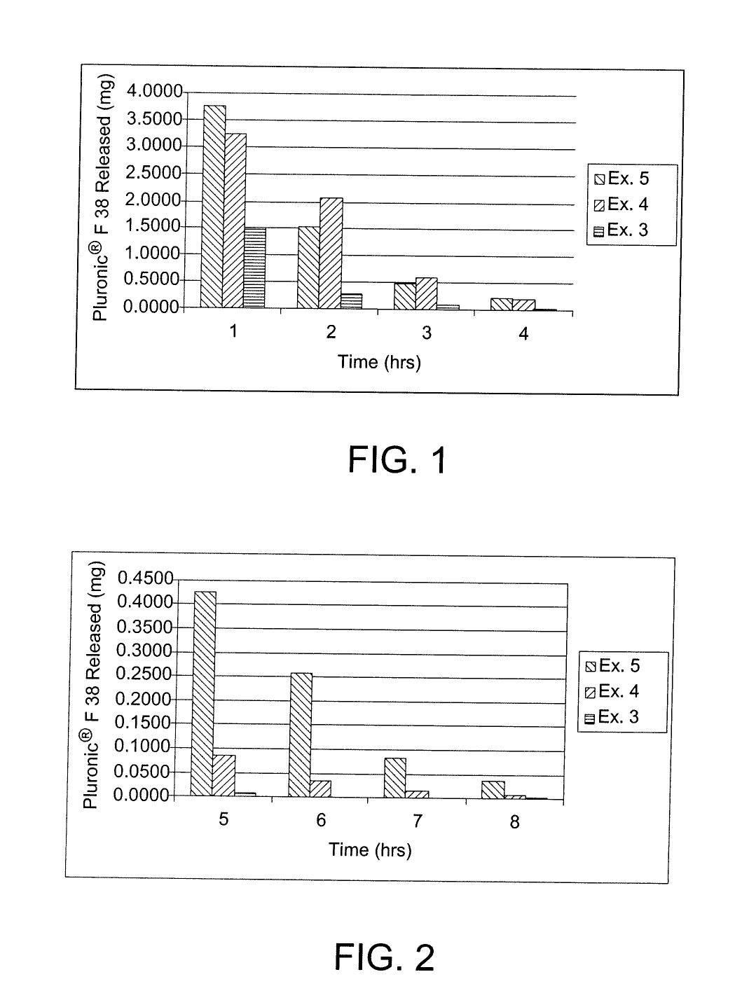

The untreated contact lens thus obtained was immersed into a 5% solution of Pluronic® F38 (HLB=31, Mw˜4700) in water in a polypropylene lens case for a period of at least 18 hours. The lens was removed from the solution and deuterium oxide (D₂O) was dripped over the surface of the lens at a rate of 1 mL/hour. At the conclusion of each hour, the D₂O was collected and ¹H NMR was used to determine the amount of Pluronic® F38 in the extract. The results for this example are shown in FIGS. 1 and 2.

### EXAMPLE 4

The untreated contact lens obtained in Example 3 was immersed into a 10% solution of Pluronic® F38 (HLB=31, Mw˜4700) in water in a polypropylene lens case for a period of at least 18 hours. The release of Pluronic® F38 over time was monitored as described above. The results for this example are shown in FIGS. 1 and 2.

### EXAMPLE 5

The untreated contact lens obtained in Example 3 was immersed into a 20% solution of Pluronic® F38 (HLB=31, Mw˜4700) in water in a polypropylene lens case for a period of at least 18 hours. The release of Pluronic® F38 over time was monitored as described above. The results for this example are shown in FIGS. 1 and 2.

### EXAMPLE 6

A monomer mixture was prepared by mixing the following components, NVP (90 weight percent); TBE (10 weight percent), DM (HLB=22, Mw˜12600) (10 weight percent), EGDMA (0.3 weight percent) and a Vazo 64 initiator (0.5 weight percent). The monomeric mixture was cast in a polypropylene contact lens mold and thermally cured for about 4 hours. The resulting contact lens had an EWC of approximately 82%.

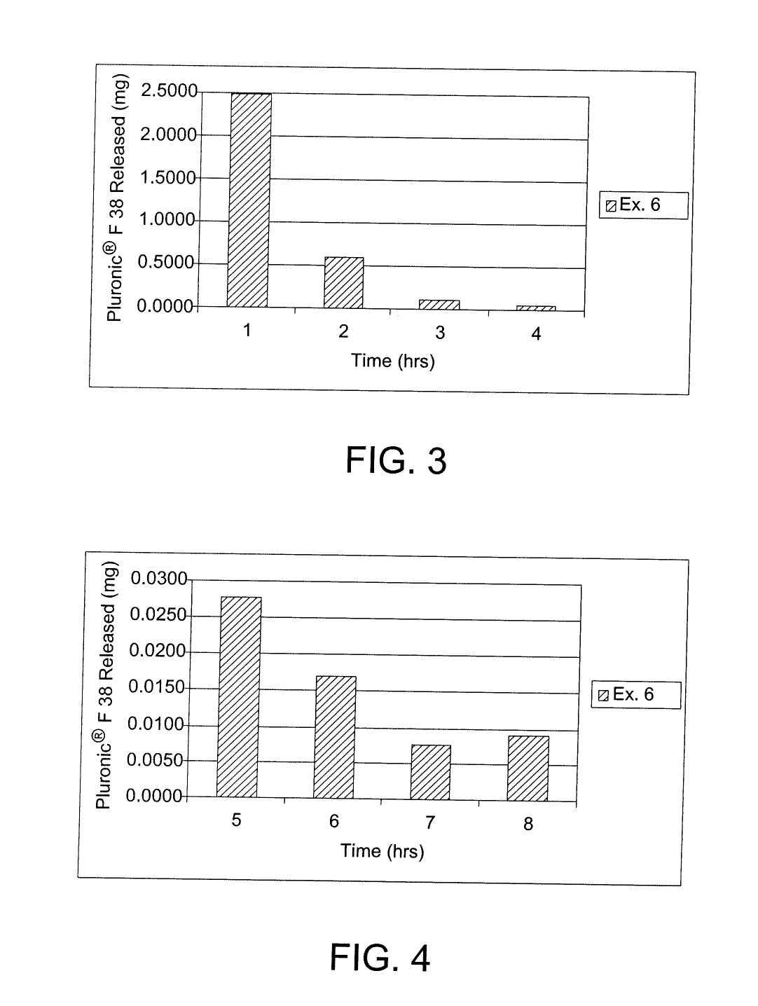

The untreated contact lens thus obtained was immersed into a 10% solution of Pluronic® F38 (HLB=31, Mw˜4700) in water in a polypropylene lens case for a period of at least 18 hours. The release of Pluronic® F38 over time was monitored as described above. The results for this example are shown in FIG. 3 and 4.

### EXAMPLE 7

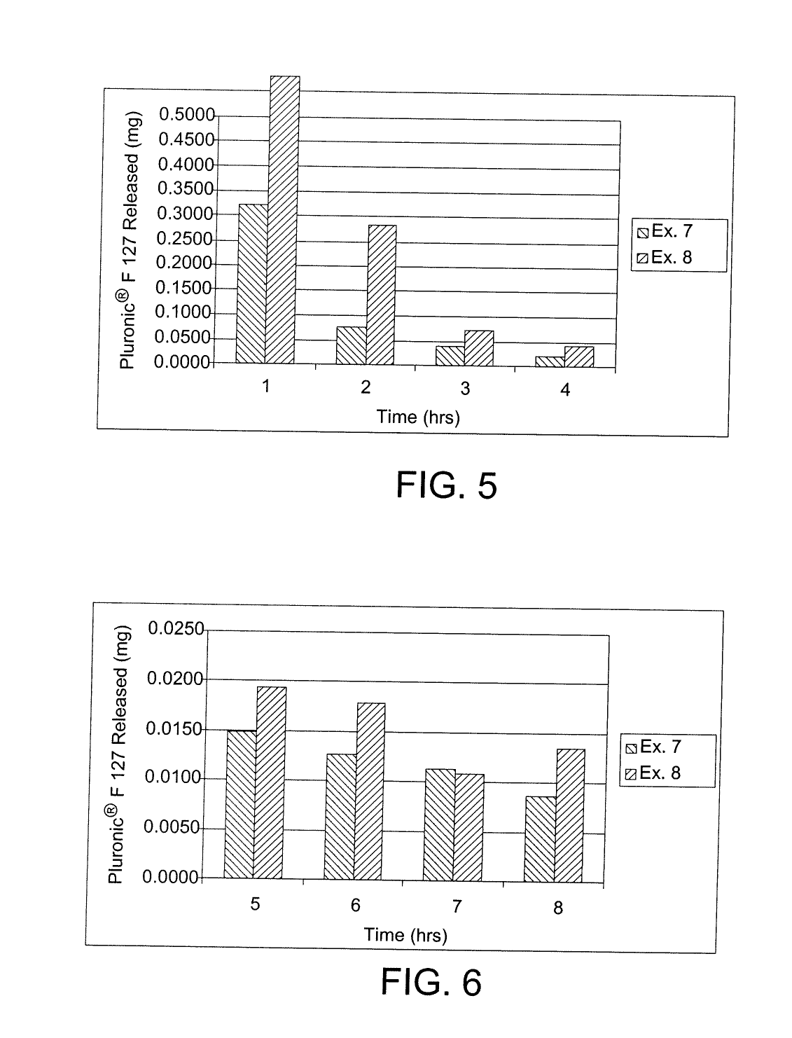

The untreated contact lens obtained in Example 6 was immersed into a 5% solution of Pluronic® F127 (HLB=22, Mw˜12600) in water in a polypropylene lens case for a period of at least 18 hours. The release of Pluronic® F127 over time was monitored as described above. The results for this example are shown in FIGS. 5 and 6.

### EXAMPLE 8

The untreated contact lens obtained in Example 6 was immersed into a 10% solution of Pluronic® F127 (HLB=22, Mw˜12600) in water in a polypropylene lens case for a period of at least 18 hours. The release of Pluronic® F127 over time was monitored as described above. The results for this example are shown in FIGS. 5 and 6.

### COMPARATIVE EXAMPLE A

A monomer mixture was prepared by mixing the following components, NVP (90 weight percent), TBE (10 weight percent), EGDMA (0.3 weight percent) and a Vazo 64 initiator (0.5 weight percent). The monomeric mixture was cast in a polypropylene contact lens mold and thermally cured for about 4 hours. The resulting contact lens had an EWC of approximately 82%.

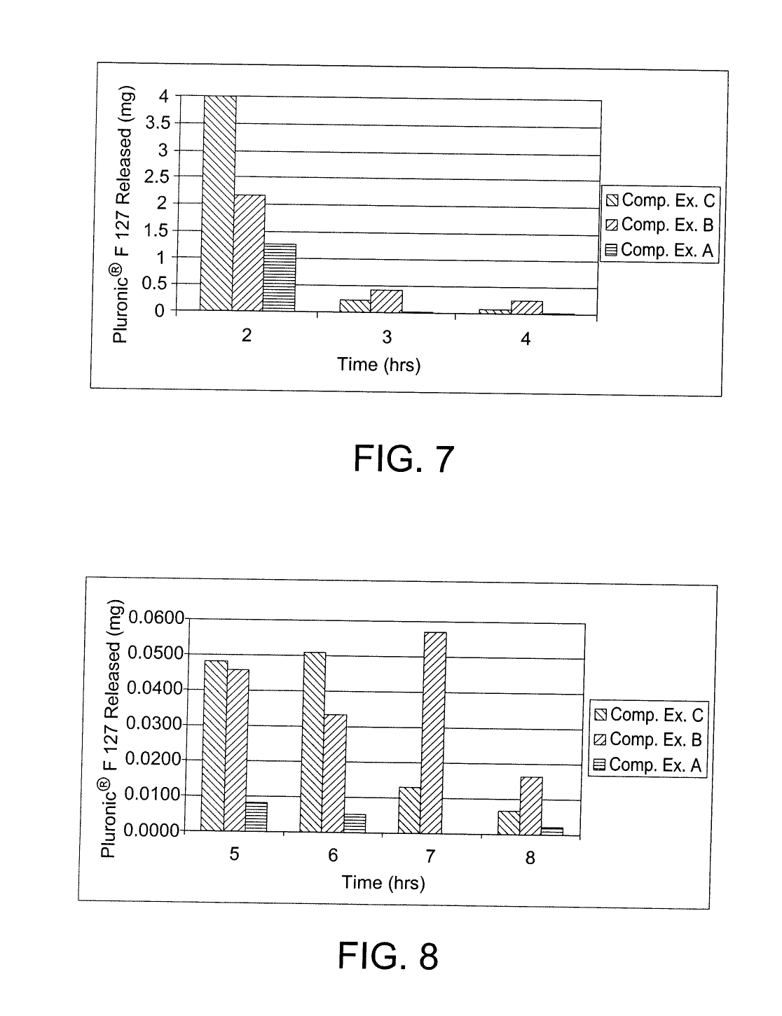

The untreated contact lens thus obtained was immersed into a 2% solution of Pluronic® F127 (HLB=22, Mw˜12600) in water in a polypropylene lens case for a period of at least 18 hours. The release of Pluronic® F127 over time was monitored as described above. The results for this example are shown in FIGS. 7 and 8.

### COMPARATIVE EXAMPLE B

The untreated contact lens obtained in Comparative Example A was immersed into a 5% solution of Pluronic® F127 (HLB=22, Mw˜12600) in water in a polypropylene lens case for a period of at least 18 hours. The release of Pluronic® F127 over time was monitored as described above. The results for this example are shown in FIGS. 7 and 8.

### COMPARATIVE EXAMPLE C

The untreated contact lens obtained in Comparative Example A was immersed into a 10% solution of Pluronic® F127 (HLB=22, Mw˜12600) in water in a polypropylene lens case for a period of at least 18 hours. The release of Pluronic® F127 over time was monitored as described above. The results for this example are shown in FIGS. 7 and 8.

### COMPARATIVE EXAMPLE D

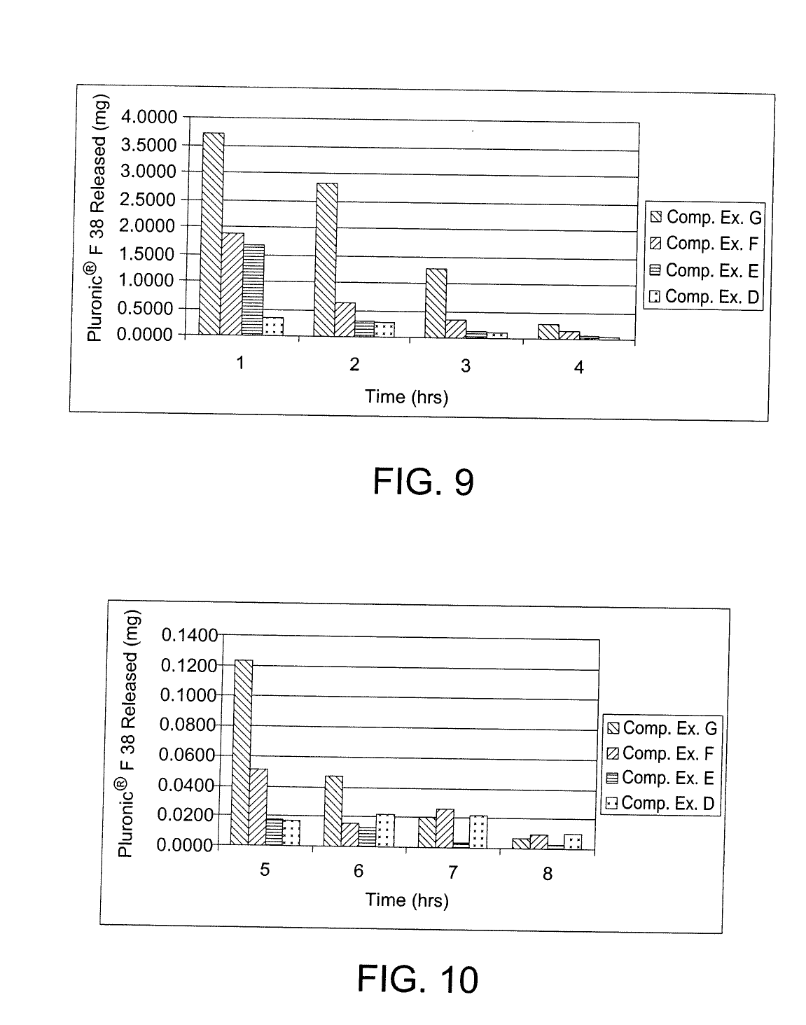

The untreated contact lens obtained in Comparative Example A was immersed into a 2% solution of Pluronic® F38 (HLB=31, Mw˜4700) in water in a polypropylene lens case for a period of at least 18 hours. The release of Pluronic® F38 over time was monitored as described above. The results for this example are shown in FIGS. 9 and 10.

### COMPARATIVE EXAMPLE E

The untreated contact lens obtained in Comparative Example A was immersed into a 5% solution of Pluronic® F38 (HLB=31, Mw˜4700) in water in a polypropylene lens case for a period of at least 18 hours. The release of Pluronic® F38 over time was monitored as described above. The results for this example are shown in FIGS. 9 and 10.

### COMPARATIVE EXAMPLE F

The untreated contact lens obtained in Comparative Example A was immersed into a 10% solution of Pluronic® F38 (HLB=31, Mw˜4700) in water in a polypropylene lens case for a period of at least 18 hours. The release of Pluronic® F38 over time was monitored as described above. The results for this example are shown in FIGS. 9 and 10.

### COMPARATIVE EXAMPLE G

The untreated contact lens obtained in Comparative Example A was immersed into a 20% solution of Pluronic® F38 (HLB=31, Mw˜4700) in water in a polypropylene lens case for a period of at least 18 hours. The release of Pluronic® F38 over time was monitored as described above. The results for this example are shown in FIGS. 9 and 10.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A method for the mitigation of symptoms of dry eye, the method comprising (a) incorporating one or more non-functionalized polymers having one or more hydrophilic moieties into an ophthalmic device that is a polymerization product of a comonomer mixture comprising: (i) a major amount of a non-silicone-containing hydrophilic monomer; and (ii) an end terminal functionalized surfactant; and (b) inserting the ophthalmic device in the eye of a patient.

2. The method of claim 1, wherein the ophthalmic device has an equilibrium water content of at least about 70 weight percent.

3. The method of claim 1, wherein the ophthalmic device has an equilibrium water content of at least about 80 weight percent.

4. The method of claim 1, wherein the non-functionalized polymer is selected from the group consisting of a polyether, polysaccharide, polyamide, polyalcohol, polylactone, polyimide, polylactam, copolymers thereof and mixtures thereof.

5. The method of claim 4, wherein the polyether is based upon poly(ethylene oxide)-poly(propylene oxide)-poly(ethylene oxide) and poly(propylene oxide)-poly(ethylene oxide)-poly(propylene oxide).

6. The method of claim 4, wherein the polyether is selected from the group consisting of a non-functionalized poloxamer, non-functionalized reverse poloxamer, non-functionalized poloxamine, non-functionalized reverse poloxamine and mixtures thereof.

7. The method of claim 1, wherein the non-functionalized polymer is selected from the group consisting of poly-N-vinyl pyrrolidone, poly(N-vinyl-N-methylacetamide), poly-N-vinyl-2-piperidone, poly-N-vinyl-2-caprolactam, poly-N-vinyl-3-methyl-2-caprolactam, poly-N-vinyl-3-methyl-2-piperidone, poly-N-vinyl-4-methyl-2-piperidone, poly-N-vinyl-4-methyl-2-caprolactam, poly-N-vinyl-3-ethyl-2-pyrrolidone, poly-N-vinyl-4,5-dimethyl-2-pyrrolidone, polyvinylimidazole, poly-N-N-dimethylacrylamide, polyvinyl alcohol, polyethylene oxide, poly 2 ethyl oxazoline, hydroxypropyl cellulose, hydroxypropyl methyl cellulose, hydroxyethyl cellulose, carboxy methylcellulose, methyl cellulose, copolymers thereof and mixtures thereof.

8. The method of claim 1, wherein the non-silicone-containing hydrophilic monomer is selected from the group consisting of an amide, cyclic lactam, poly(alkene glycols) functionalized with polymerizable groups and mixtures thereof.

9. The method of claim 1, wherein the non-silicone-containing hydrophilic monomer is selected from the group consisting of N,N-dimethylacrylamide, N,N-dimethylmethacrylamide, N-vinyl-2-pyrrolidone and mixtures thereof.

10. The method of claim 1, wherein the end terminal functionalized surfactant is selected from the group consisting of an end terminal functionalized poloxamer, end terminal functionalized reverse poloxamer, end terminal functionalized poloxamine, end terminal functionalized reverse poloxamine and mixtures thereof.

11. The method of claim 1, wherein the non-functionalized polymer is selected from the group consisting of a non-functionalized poloxamer, non-functionalized reverse poloxamer, non-functionalized poloxamine, non-functionalized reverse poloxamine and mixtures thereof and the end terminal functionalized surfactant is selected from the group consisting of an end terminal functionalized poloxamer, end terminal functionalized reverse poloxamer, end terminal functionalized poloxamine, end terminal functionalized reverse poloxamine and mixtures thereof.

12. The method of claim 1, wherein the end terminal functionalized surfactant is present in the comonomer mixture in an amount of about 0.01 to about 20 weight percent, based on the total weight of the comonomer mixture.

13. The method of claim 1, wherein the non-functionalized polymer is selected from the group consisting of a non-functionalized poloxamer, non-functionalized reverse poloxamer, non-functionalized poloxamine, non-functionalized reverse poloxamine and mixtures thereof and the end terminal functionalized surfactant is selected from the group consisting of an end terminal functionalized poloxamer, end terminal functionalized reverse poloxamer, end terminal functionalized poloxamine, end terminal functionalized reverse poloxamine and mixtures thereof.

14. The method of claim 1, wherein the comonomer mixture further comprises a hydrophobic monomer.

15. The method of claim 1, wherein the hydrophobic monomer is selected from the group consisting of a (meth)acrylate-containing hydrophobic monomer, N-alkyl(meth)acrylamide-containing hydrophobic monomer, alkyl vinylcarbonate-containing hydrophobic monomer, alkyl vinylcarbamate-containing hydrophobic monomer, fluoroalkyl(meth)acrylate-containing hydrophobic monomer, N-fluoroalkyl(meth)acrylamide-containing hydrophobic monomer, N-fluoroalkyl vinylcarbonate-containing hydrophobic monomer, N-fluoroalkyl vinylcarbamate-containing hydrophobic monomer, silicone-containing (meth)acrylate-containing hydrophobic monomer, (meth)acrylamide-containing hydrophobic monomer, vinyl carbonate-containing hydrophobic monomer, vinyl carbamate-containing hydrophobic monomer, styrenic-containing hydrophobic monomer, polyoxypropylene(meth)acrylate-containing hydrophobic monomer and mixtures thereof.

16. The method of claim 1, wherein the step of incorporating comprises contacting the ophthalmic device with a solution comprising the one or more non-functionalized polymers for a time period sufficient to incorporate the non-functionalized polymer into the device.

17. The method of claim 16, wherein the non-functionalized polymer is selected from the group consisting of a polyether, polysaccharide, polyamide, polyalcohol, polylactone, polyimide, polylactam, copolymers thereof and mixtures thereof.

18. The method of claim 17, wherein the non-functionalized polyether is based upon poly(ethylene oxide)-poly(propylene oxide)-poly(ethylene oxide) and poly(propylene oxide)-poly(ethylene oxide)-poly(propylene oxide).

19. The method of claim 16, wherein the non-functionalized polymer is selected from the group consisting of a non-functionalized poloxamer, non-functionalized reverse poloxamer, non-functionalized poloxamine, non-functionalized reverse poloxamine and mixtures thereof.

20. The method of claim 16, wherein the non-functionalized polymer is present in the solution from about 0.001 to about 20% by weight of the solution.

21. The method of claim 1, wherein the ophthalmic device is a contact lens.

22. A method for the mitigation of symptoms of dry eye, the method comprising (a) providing an ophthalmic device that is a polymerization product of a comonomer mixture comprising: (i) a major amount of a non-silicone-containing hydrophilic monomer; a major amount of a non-silicone-containing hydrophilic monomer; and (ii) an end terminal functionalized surfactant; (b) contacting the ophthalmic device with a solution comprising one or more non-functionalized polymers having one or more hydrophilic moieties for a time period sufficient to incorporate the one or more non-functionalized polymers having one or more hydrophilic moieties into the ophthalmic device; and (c) inserting the ophthalmic device in the eye of a patient.

23. The method of claim 22, wherein the non-functionalized polymer is selected from the group consisting of a non-functionalized poloxamer, non-functionalized reverse poloxamer, non-functionalized poloxamine, non-functionalized reverse poloxamine and mixtures thereof and the end terminal functionalized surfactant is selected from the group consisting of an end terminal functionalized poloxamer, end terminal functionalized reverse poloxamer, end terminal functionalized poloxamine, end terminal functionalized reverse poloxamine and mixtures thereof.

24. The method of claim 22, wherein the end terminal functionalized surfactant is present in the comonomer mixture in an amount of about 0.01 to about 20 weight percent, based on the total weight of the comonomer mixture.

25. The method of claim 22, wherein the non-functionalized polymer is present in the solution from about 0.001 to about 20% by weight of the solution.
